(19) United States
(12) Patent Application Publication
(10) Pub. No.: US 2021/0308414 A1
(43) Pub. Date: Oct. 7, 2021

(54) Clothing for self-stimulatory action ("stim") and related methods

(71) Applicant: Randall Foster, La Mesa, CA (US)

(72) Inventor: Randall Foster, La Mesa, CA (US)

(21) Appl. No.: 17/222,794

(22) Filed: Apr. 5, 2021

Related U.S. Application Data
(60) Provisional application No. 63/006,001, filed on Apr. 6, 2020.

(51) Int. Cl. A61M 21/02; A41B 1/08

(52) U.S. Cl. CPC A61M 21/02; A61M 2021/0022; A41B 1/08

(57) ABSTRACT

Disclosed is clothing subtly outfitted with tactile stimulation objects to provide stress relief and relaxation to individuals who are Autistic or suffer from a stress or anxiety disorder. The tactile stimulation objects come in the form of packaged objects sewn into different areas on the clothing.

### CROSS-REFERENCE TO RELATED APPLICATIONS

Not applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO AN APPENDIX SUBMITTED ON A COMPACT DISC AND INCORPORATED BY REFERENCE OF THE MATERIAL ON THE COMPACT DISC

Not applicable.

### STATEMENT REGARDING PRIOR DISCLOSURES BY THE INVENTOR OR A JOINT INVENTOR

Reserved for a later date, if necessary.

### BACKGROUND OF THE INVENTION

### Field of Invention

The disclosed subject matter is in the field of clothing for self-stimulatory action (“stim’) and tactile stimulation objects used in connection with adaptive clothing.

### Background of the Invention

Tactile stimulation is the process of activating a plurality of nerve signals beneath the skins surface which inform the body of sensory information such as texture, temperature, and other tactile sensations. These sensations can soothe (i.e., otherwise provide comfort or an emotional sense of peace and stress-relief) to autistic, fidgety, or neurotypical persons suffering from lack of stimulation. Such persons that are appeased by tactile stimulation have, what are classified as, tactile sensory needs.

A person with tactile sensory needs has a tendency to stim (i.e., take repetitive self-stimulatory action (such as flapping the hands, bounding a leg, spin in a chair, fingernail bite, and the like)). But, uncontrolled stimming can be embarrassing. So, often those with tactile sensory needs need a tactile stimulation object that is designed for discrete stimming. These objects can be nearly anything, but generally are small enough to be easily carried and may be used to create a tactile sensation. Some of these objects are designed for tactile stimulation while others are simply objects with unique and stimulating features. For example, such an object may be naturally occurring, such as a pinecone, or a manufactured object such as a fidget spinner. The ideal object for any particular person is determined by the needs and preferences of that person.

It is sometimes the case that a person with tactile sensory needs can become fidgety when such a tactile stimulation object is not readily available. Under such circumstances, these individuals have been known to use their shirts in different ways to meet those needs if there are no other options available. Usually, articles of clothing are not satisfactory tactile stimulation objects because they lack a unique texture necessary for stimulation, lack the requisite durability, or are conspicuous. Using clothing to satisfy these sensory needs can be embarrassing or unsatisfying to the person in need of stimulation and damaging to the person's clothing.

Sometimes, tactile sensory needs are the result of underlying medical conditions (e.g. Autism) or are children looking for stimulation. It is important that the care-takers of those persons (like parents, teachers, and guardians) ensure access to tactile stimulation objects if they are necessary for the individual to relax. Although these objects are primarily useful for those with Autism, it is also important that other populations have access to these objects. Individuals that struggle with chronic or acute anxiety may also benefit from a system that allows for self-soothing via tactile stimulation in an inconspicuous and readily-accessible manner. Without access to such objects, individuals in need are left uncomfortable, bothered, or anxious.

Often, individuals with tactile sensory needs are limited to tactile stimulation objects that are detached or separate from their clothing. This is a problem because these small objects are inherently easy to lose, drop, or break. When one of these tactile objects is not accessible, individuals may start to grab at improper objects to provide the stimulation they need.

The problem of lost or inaccessible tactile stimulation objects will continue to plague caretakers, teachers, and parents of those with tactile needs until a tool or method is created which integrates tactile stimulation objects and clothing. This problem is compounded by the fact that, even when these tactile stimulation objects are accessible, those with tactile needs may be reprimanded or ostracized by oblivious individuals for inappropriate use of tactile stimulation objects.

Thus, a need exists for an article of clothing which has tactile stimulation objects inconspicuously integrated into it so that such objects are subtle and readily accessible.

### PRIOR ART

Current adaptive clothing technologies that exist for the autistic or anxiety disorder population is limited to both seamless articles and soft fabrics. Another disclosed concept is one that places these sorts of self-stimulating items on top of articles of clothing. However, these concepts fail to address the needs of the Autistic population because these configurations leaves stimulation objects susceptible to being broken or lost. Further, these configurations are not adequately inconspicuous.

Other approaches to adaptive clothing in this space is Target Corporation's “Cat & Jack” clothing line. This line is considered adaptive because it features seamless clothing, dual-fabric material (cotton and polyester), no tags, and reinforced stitching of the knee portion of denim jeans.

US20150273178A1 discloses, “Every day clothing integrated with sensory therapy and on board fidgets.”

### SUMMARY OF THE INVENTION

In view of the foregoing, an object of this specification is to disclose an article of clothing that has tactile stimulation objects or “stimming technology” built into the fabric for discrete and on-demand stimming by the wearer or others close to the wearer.

It is another object of this specification to disclose an article of clothing with stimming technology hidden or permanently stored inside the fabric.

It is yet another object of this specification to disclose an article of clothing that reduces the chance of the stimulation object being lost or destroyed.

It is yet another object of this specification to disclose an article of clothing that allows for inconspicuous stimming.

It is yet another object of this specification to disclose a durable article of clothing that can endure stimming.

It is yet another object of this specification to provide an emotional sense of peace and relief from stress by allowing an individual with sensory tactile needs (e.g. those with autism) to stim with the stimming technology that is placed into articles of clothing in an inconspicuous and comforting manner.

Other objectives of the invention will become apparent to those skilled in the art once the invention has been shown and described. These objectives are not to be construed as limitations of applicant's invention, but are merely aimed to suggest some of the many benefits that may be realized by the apparatus of the present application and with its many embodiments.

### BRIEF DESCRIPTION OF THE SEVERAL VIEWS OF THE DRAWINGS

Other objectives of the disclosure will become apparent to those skilled in the art once the invention has been shown and described. The manner in which these objectives and other desirable characteristics can be obtained is explained in the following description and attached figures in which:

FIG. 1 is an environmental view of a preferred embodiment of a t-shirt with integrated tactile stimulation objects;

FIG. 2 is an environmental view of a preferred embodiment of the t-shirt with emphasis on details of the tactile stimulation objects;

FIG. 3 is a front view of the t-shirt;

FIG. 4 is a side view of the t-shirt;

FIG. 5 is a top view of the t-shirt.

In the figures, each component of the t-shirt shown in the figures is labeled and indicated by the numeral as shown on the list below;
1—t-shirt;
2—packages;
2a—tactile stimulation objects or materials;
3—waist hem line;
4—shoulder hem line;
5—collar hem line; and,
6—yoke hem line.

It is to be noted, however, that the appended figures illustrate only typical embodiments of this invention and are therefore not to be considered limiting of its scope, for the invention may admit to other equally effective embodiments that will be appreciated by those reasonably skilled in the relevant arts. Also, figures are not necessarily made to scale but are representative.

### DETAILED DESCRIPTION OF PREFERRED EMBODIMENTS

Disclosed is clothing with built-in stimming technology or other tactile sensory objects. Clothing may include sweaters, pajamas, shorts, pants, coats and any article of clothing with a hem or seam. A preferred embodiment of the disclosed clothing is a t-shirt 1. The details of the disclosed clothing can be ascertained via a description of the preferred embodiment and reference to the figures.

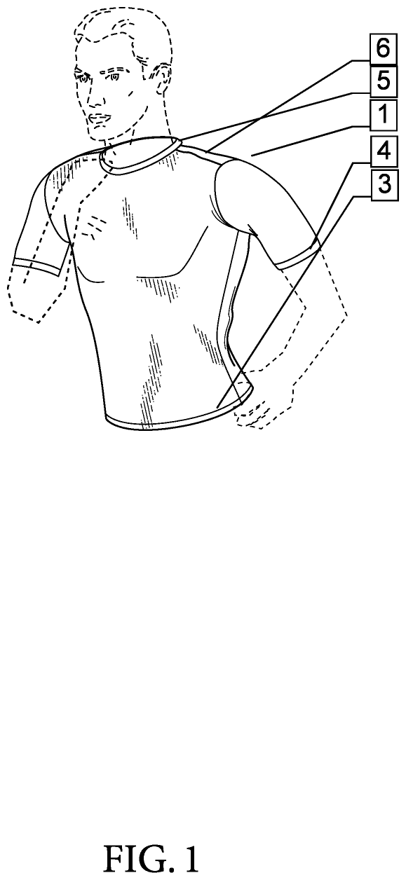

FIG. 1 is an environmental view of the preferred embodiment of a t-shirt 1. As shown, the t-shirt 1 is made of fabric sewn into two sleeves, a neckline, and a hem. Aside from the augmentation of tactile stimulation objects in the form of a plurality of packages 2, this t-shirt is similar to a traditional t-shirt. As shown in FIG. 1, the t-shirt 1 covers the chest, stomach, upper arms, back, and shoulders of the individual shown.

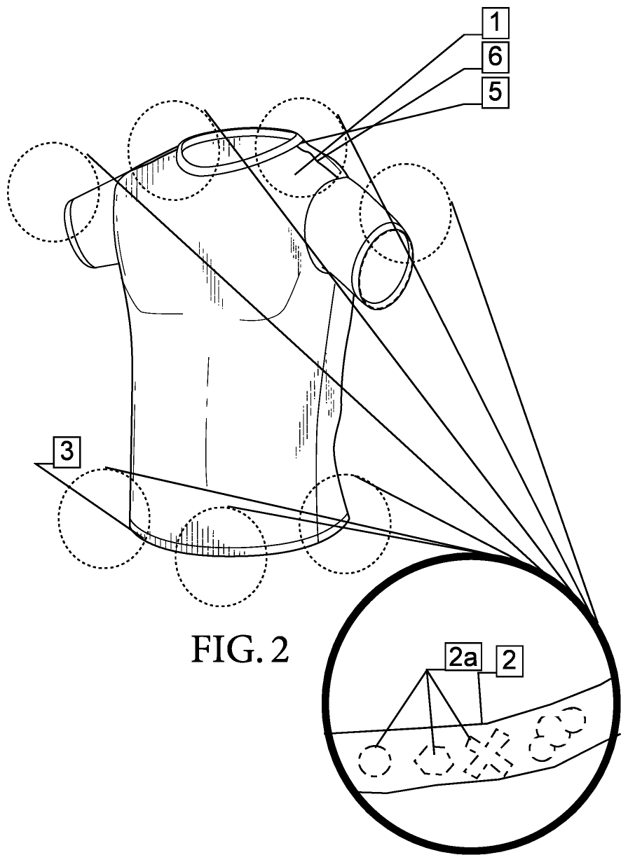

FIG. 2 is an environmental view of the preferred embodiment of the t-shirt 1 with enlarged detail to emphasize integrated packages or folds that house or hide tactile stimulation objects 2a. Although the figures focus on augmentation to a t-shirt 1, tactile stimulation objects 2a may be added to any article of clothing in this manner (e.g., by sewing into the hem or fold of the clothing). As shown, stimulation objects are placed inside the hemlines of the shirt. The objects 2a are sized and placed so that they can be used discreetly. This embodiment features discreet object 2a placement in the waist hemline 3, shoulder hemlines 4, yoke hemline 6, and/or collar hemline 5. The enlarged detail shows the different objects 2a which may be placed inside the hemlines of the t-shirt 1. When sewn into the hemline, the objects 2a in housed in a compartment or pocket in the form of packages. The packages 2 may be permanent or removable and replaceable within a pocket or compartment sewn into the hem.

The stimulation objects may be small packages 2 filled with different materials sewn into youth and adult sized t-shirts 1. Preferably, the packages 2 may be sewn into the waist hems 3 on the left and right sides of t-shirts 1, preferably by the hips, and into the sleeve hemlines on the left and right sides of the t-shirts 1. However, the packages 2 may also be sewn into the collar hemline 5 of the t-shirt 1, or into a pocket. Essentially, the packages 2 may be sewn and integrated anywhere in the shirt.

The placement and composition of packages is determined by the needs of the individual. The t-shirts 1 may be customized for specific needs or mass produced for an entire population. T-shirts 1 also may be augmented aftermarket or during manufacture of the original t-shirt 1.

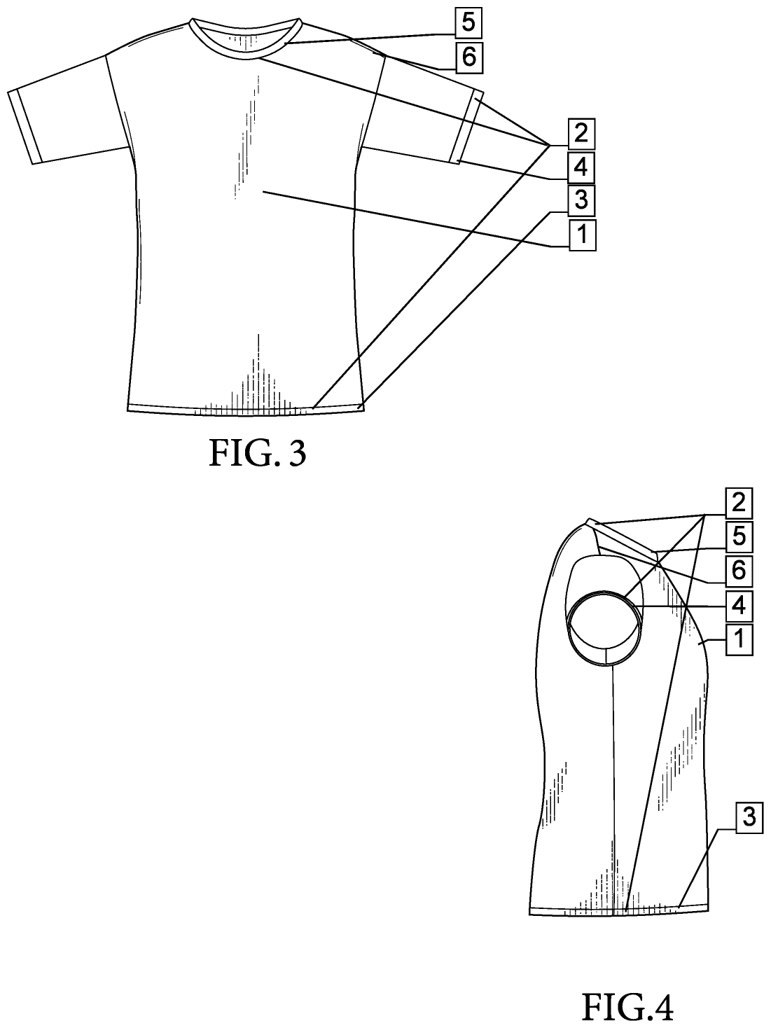

FIGS. 3 and 4 show a front and side view of the t-shirt 1. As shown, the packages 2 may be preferably sewn into any hemline, or otherwise anywhere else on the t-shirt 1. Although packages 2 may be integrated into any part of the shirt, or anywhere on any other article of clothing, hemlines are preferred. Hemlines are a preferred location for the packages 2 because hemlines are inherently more bulky, and therefore, reinforced. Hemlines tend to be the most durable area on an article of clothing because they feature multiple layers of fabric on top of each other.

Hemlines also tend to be in more discreet places on the t-shirt 1 because they are considered visually unappealing. This is convenient for package 2 placement as it is important that packages 2 are placed in discreet areas. Because of the extra hemline sewing inherent in manufacturing the shirt, package placement may be easily integrated to manufacturing processes already in place. Discreet package placement is important because individuals with tactile stimulation needs will likely need to stim at inappropriate times. So, discreet locations for packages 2 is ideal.

Further, most hemlines are easily accessed by the hands of the wearer. The waist hemline 3 is normally inches away from the hands of the wearer when an arm of an individual is in a relaxed position. Since most interactions are face to face specific more discreet parts of hemlines may be more desirable locations for packages 2 than others. Specifically, this makes the sides of the waist 3 hemline preferable to other locations of the waist 3 hemline. For the same reasons it may also be advantageous to place packages on the bottom side of the shoulder hemline 5 than the top.

The preferred packages 2 which may be placed in the hemlines of the shirt may be white ⅝ inch compression finger tubular bandages which may be used to package unique nonhomogeneous material which may provide tactile stimulation to a wearer. These type of packages 2 are preferred because of they are relatively thick, enduring, washable, and properly sized. Although many different pieces of material 2a may be stored in the packages 2, the packages 2 may be preferably filled with 1 cm flat round marbles, 2 cm plastic prism gems, ½ cm plastic octagonal beads, 1 cm round plastic spheres/marbles, 2 cm glass marbles, memory foam, and gel. The preferred sewing thread for placing the packages 2 is a polyester thread.

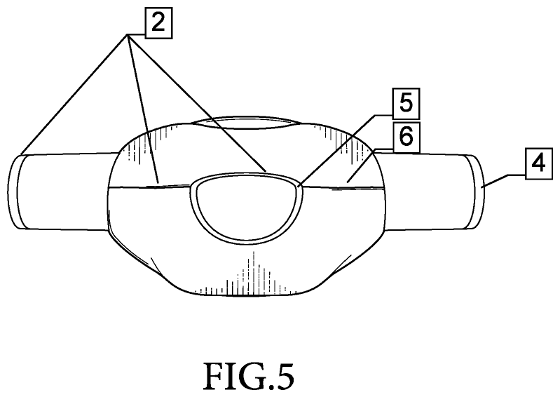

FIG. 5 shows a top view of the t-shirt 1. Shown in the figure are different hemlines which may be augmented with packages 2 for tactile stimulation. The packages 2 preferably made out of compression tubular bandages filled with stimulating materials and sewn closed. The process by which such packages 2 may be made and placed on or in the t-shirt 1 begins with a sewing machine. Then one must acquire the necessary packages 2 and material to store in the packages 2. One may sew one side of the package closed and through the open side, insert as much material as deemed necessary to provide adequate stimulation. The packages 2 may also be closed by employing other means of adhesion such as glue, or by cauterizing the packages closed. The packages 2 must be filled with enough material to stimulate but not so much that the package is overburdened or that the materials are lodged in place. Generally, the preferred amount of material is 10-50 pieces of material. However, the ideal number of pieces varies by type since different materials have different sizes. The length of the compression tubular bandages when filled with material may be between 4 cm to 20 cm in length and varies due to material type, shirt size, and package location. One may sew the package closed once the ideal amount of material is added to the package.

FIG. 5 also shows a shoulder hemline 4 and a yoke hemline 6 that is augmented with packages 2. The complete packages 2 are placed once the packages 2 are sewn closed. To sew the packages 2 into the hem one may place the packages 2 on the bottom portion of an open hem. Then, one may fold the shirt up 1-2 inches to cover the package. Then, one may sew a new hemline into the shirt using a sewing machine, all the way around the shirt, in order to seal in the packages 2. To add packages 2 to new hemlines one may simply repeat the aforementioned process as many times as necessary.

Although these features and methods are described in terms of a t-shirt it is understood that those skilled in the art could apply different stimulation objects to different articles of clothing. Other articles include, but are not limited to, pants, long sleeved shirts, sweat shirts, vests, jackets, pants, and the like.

Although the method and apparatus is described above in terms of various exemplary embodiments and implementations, it should be understood that the various features, aspects and functionality described in one or more of the individual embodiments are not limited in their applicability to the particular embodiment with which they are described, but instead might be applied, alone or in various combinations, to one or more of the other embodiments of the disclosed method and apparatus, whether or not such embodiments are described and whether or not such features are presented as being a part of a described embodiment. Thus the breadth and scope of the claimed invention should not be limited by any of the above-described embodiments.

Terms and phrases used in this document, and variations thereof, unless otherwise expressly stated, should be construed as open-ended as opposed to limiting. As examples of the foregoing: the term “including” should be read as meaning “including, without limitation” or the like, the term “example” is used to provide exemplary instances of the item in discussion, not an exhaustive or limiting list thereof, the terms “a” or “an” should be read as meaning “at least one,” “one or more,” or the like, and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that might be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases might be absent. The use of the term “assembly” does not imply that the components or functionality described or claimed as part of the module are all configured in a common package. Indeed, any or all of the various components of a module, whether control logic or other components, might be combined in a single package or separately maintained and might further be distributed across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives might be implemented without confinement to the illustrated examples. For example, block diagrams and their accompanying description should not be construed as mandating a particular architecture or configuration.

All original claims submitted with this specification are incorporated by reference in their entirety as if fully set forth herein.

We claim:

1. An article of clothing including at least one hemline into which a tactile sensory object has been sewn.

2. A method of treating a person with tactile sensory needs comprising the step of putting an article of clothing on the person and letting the person stim any one of a plurality of hidden stimming technologies provided within the garment.

3. A method of manufacturing an article of clothing comprising the step of sewing a hemline that hides at least one tactile sensory object therein the hemline.
